(19) United States
(12) Patent Application Publication
(10) Pub. No.: US 2016/0002717 A1
(43) Pub. Date: Jan. 7, 2016

(54) DETERMINING MUTATION BURDEN IN CIRCULATING CELL-FREE NUCLEIC ACID AND ASSOCIATED RISK OF DISEASE

(71) Applicant: Boreal Genomics, Inc., Vancouver (CA)

(72) Inventors: Mark Lee, Los Altos Hills, CA (US); Andrea Marziali, North Vancouver (CA); Matthew Wiggin, Vancouver (CA); Joel Pel, Vancouver (CA); Nitin Sood, Los Altos, CA (US)

(21) Appl. No.: 14/322,523

(22) Filed: Jul. 2, 2014

(51) Int. Cl. C12Q 1/68

(52) U.S. Cl. CPC C12Q 1/6869; C12Q 1/6874

(57) ABSTRACT

The invention generally relates to methods of assessing an individual's risk of developing a disease associated with accumulation of DNA mutations by determining the mutation burden in their circulating cell-free nucleic acid relative to a reference sequence. The invention further relates to establishing a score indicative of the individual's risk by assessing the individual's mutation burden against a mutation burden continuum containing various thresholds associated with different degrees of risk. The reference sequence and the continuum may be constructed from a variety of sources. In certain aspects, methods of the invention relate to compilation of a database of mutation burdens for individuals along with population characteristics for each individual.

### FIELD OF THE INVENTION

The invention generally relates to methods for determining a risk of developing a disease associated with accumulation of DNA mutations based on analysis of mutation burden in circulating cell-free nucleic acid.

### BACKGROUND

An individual accumulates somatic mutations throughout life. Somatic mutations can result from a variety of causes, including exposure to ultraviolet radiation, chemical exposure, diet, or other environmental sources. The accumulation of somatic mutations has been associated with the development of several diseases, including metabolic diseases, cancer, neurological diseases, autoimmune disorders, and cardiovascular diseases. In particular, cancer is thought to be a disease associated with accumulated genomic instability, which may arise through a combination of environmental exposures and intrinsic host susceptibility to somatic mutation (e.g., due to variability in efficiency of DNA repair mechanisms).

In many cases, early detection greatly improves treatment options and outcomes. Screening regimens for many diseases are expensive and may involve invasive procedures that carry risks to the individual. Accordingly, some screening procedures are recommended only to certain populations. Those recommendations are typically selected based on factors such as age, fitness level, behavioral history (e.g., smoking or diet), and family history. However, screening typically is not tailored to individuals and generalized risk profiling may result in unnecessary screening or failure to screen when necessary. Accordingly, there is a need in the art for methods of assessing disease risk that are tailored to individual needs.

### SUMMARY

The invention provides methods for establishing an individual's risk of developing a disease by assessing mutation burden in cell-free circulating nucleic acid. The results of methods of the invention enable a tailored screening or treatment regimen for an individual based on the individual's risk of disease. The results may also identify significant exposure to mutagenic stress and may be used to initiate further investigation into the individual's environment or lifestyle. Methods of the invention may also be coordinately assessed along with clinical, genetic, and lifestyle factors which may affect mutation burden, such as age, smoking history, family history, germline mutations and other factors in order to accurately assess an individual's likelihood of developing certain diseases.

According to methods of the invention, an individual's mutation burden is determined in isolated circulating cell-free nucleic acid from blood or another body fluid. In a preferred embodiment, the circulating cell-free nucleic acid is one or more fragments of deoxyribonucleic acid (DNA) and is isolated from a body fluid sample, preferably urine or blood plasma or serum using any of a variety of techniques known in the art. Isolated cell-free nucleic acid is amplified using polymerase chain reaction (PCR) techniques and sequenced using next generation sequencing (NGS) or other sequencing systems and techniques known in the art. In some embodiments, cell-free DNA (cfDNA) may be ligated to barcoded adaptors in order to improve the accuracy of the sequencing. In other embodiments, the random nature of the cfDNA end sequences may be used to improve sequencing accuracy.

Sequence data obtained from circulating cell-free nucleic acid is compared to a reference nucleic acid sequence in order to determine the presence and/or level of mutations, such as single nucleotide variants, deletion (including loss of heterozygosity), amplifications, insertions, rearrangements, or translocations. In certain aspects, the presence and level of mutations in an individual's cfCNA may be used to assess somatic mosaicism within the individual (i.e., where somatic cells of the individual are of more than one genotype). The mutation burden is an assessment of the overall presence of mutations in the cell-free DNA, and may be weighted in order to give more significance to one or more mutations known to be associated with a particular disease or condition. For example, a mutation in a gene with known links to a particular cancer (or cancers) may carry a higher weight than a mutation in a gene with no such links. In another example, a mutation known to be an activating mutation related to cancer, may carry a higher weight than a mutation in a tumor suppressor gene, since a remaining healthy copy of such a gene could allow a cell to work normally. Weighting may consist of assigning a multiplier to certain mutations. Methods of the invention contemplate algorithms for assessing mutation burden and for creating a score that is used to assess the risk of disease. This score may be related continuously to a quantitative risk of disease, and may be represented, for clinical purposes, either as a continuous measure or as a categorical measure (e.g., low, intermediate, high).

Circulating cell-free nucleic acid is released into blood through a variety of pathways, including apoptosis, cell lysis, breakdown of blood cells, tumor necrosis, and spontaneous release of nucleic acids; and may be isolated from plasma, cellular fractions (often in interstitial fluid or bound to cells), or exosomes. Circulating cell-free DNA may originate from any cell type located anywhere in the body.

In certain embodiments, a reference nucleic acid sequence may be determined from nucleic acid extracted from one or more somatic cells of the individual being tested. Somatic cells may be obtained through buccal swab or other techniques and may be any non-germ cell of the body, including, for example, white blood cells. Different cellular populations are subjected to varying amounts of mutagenic stress and therefore will accumulate different mutations and at different rates. For example, cells of the epidermis are exposed to more ultraviolet radiation than other cell populations. Taking advantage of this, certain aspects of the invention utilize nucleic acids from specific cell populations in order to form the reference sequence from which to determine mutation burden.

In certain aspects, a plurality of cell types may be used as a reference. The mutation burden may be calculated based upon comparing cell-free nucleic acid to an aggregate of reference sequence obtained from several cell populations. In certain aspects, the reference sequence may be determined from the cell-free nucleic acid sample. For example, where there is variability among multiple sequencing reads of the same section of an individual's cell-free nucleic acid sample, a reference sequence may be determined from the most prevalently represented sequence variation at any given locus of the individual's cell-free nucleic acid sequence.

In certain embodiments, a reference nucleic acid sequence may be a nucleic acid sequence from any cell or circulating cell-free nucleic acid which has been obtained from the individual and/or sequenced at an earlier time. For example, a sequence of DNA obtained from a buccal swab of the individual during adolescence may then be used throughout the individual's life as a non-mutated reference from which to determine present day mutation burden. Mutation burden is then used to predict the likelihood of disease or disease onset. Diseases, such as cancer, diabetes, dementia, multiple sclerosis, lupus, Parkinson's and others, are targets of methods disclosed herein. Many of these diseases may be associated with aging, however, it is not necessary that methods of the invention be conducted on aged individuals, however, as the diseases described above can affect individuals at any age and may be due to the accumulation of genetic alterations over time, which varies from individual to individual. In other embodiments, the reference may simply be the known human genome sequence (non-mutated) with a mutation frequency estimated by the average frequency in a large population of unaffected individuals. The reference sequence may also be obtained from consensus sequences available in numerous databases and from sources known in the art.

Certain aspects of the invention include, after calculating a mutation burden or weighted mutation burden for the individual, establishing a score indicative of risk of developing a disease by assessing the individual's mutation burden against a mutation burden continuum which may contain various thresholds associated with different degrees of risk. In certain embodiments, the continuum may contain average mutation burdens for one or more sample populations. A sample population may be defined by one or more population characteristics including, age, gender, race, geographic location, disease state, diet, weight, height, or other body measurements, lifestyle, or health indicators.

In certain aspects, methods of the invention relate to creating a database (e.g., through retrospective analysis of banked specimens or prospectively enrolled trials, including registries) of patient information including mutation burden and population characteristics as described above. Such a database may be used to develop and validate a model for the relationship of mutation burden (e.g., the score) to clinical outcomes (e.g., occurrence of disease within a certain timeframe) in certain embodiments of the invention. Additionally the database may be used to identify and/or track populations defined by mutation burden and other individual patient characteristics to further refine these models and to evaluate the impact of interventions (e.g., screening) tailored to these populations.

According to methods of the invention, the mutation burden for an individual may be recorded over time at multiple chronological points. This record may be used to track the accumulation of mutation burden over time and/or the change in rate of accumulation. The change in mutation burden over time may be plotted and/or used to determine a secondary risk score wherein an increase in mutation burden or an increase in the rate of mutation burden accumulation is indicative of an increased risk of developing a disease. In certain aspects, one or more of the individual's prior mutation burden scores may serve as a reference wherein the individual's risk score is determined by comparing the present mutation burden to the prior mutation burden. A chronological record of the individual's mutation burden may be used to identify sources of exposure to mutagenic stress or to identify deficiencies or deterioration in the nucleic acid repair mechanisms in the individual's cells.

The invention contemplates an algorithm for determining a risk factor for disease, which includes cfDNA mutation burden, but also includes more conventional risk factors including, but not limited to, germline mutations known to be linked to disease, age, smoking history, family history, gender, childbearing, known radiation exposure, known chemical exposure, ethnicity, number of sunburns, sunlight exposure, and similar hereditary or environmental factors. The risk factor may also weigh various somatic mutations differently based on their utility in correlating the risk factor metric to disease.

In certain embodiments the score is a numerical value wherein a higher number is indicative of an increased risk of developing the disease. In some aspects, scores of certain values may trigger a recommendation of increased screening for the disease.

### BRIEF DESCRIPTION OF THE DRAWINGS

FIG. 1 is diagram of methods of the invention.

### DETAILED DESCRIPTION

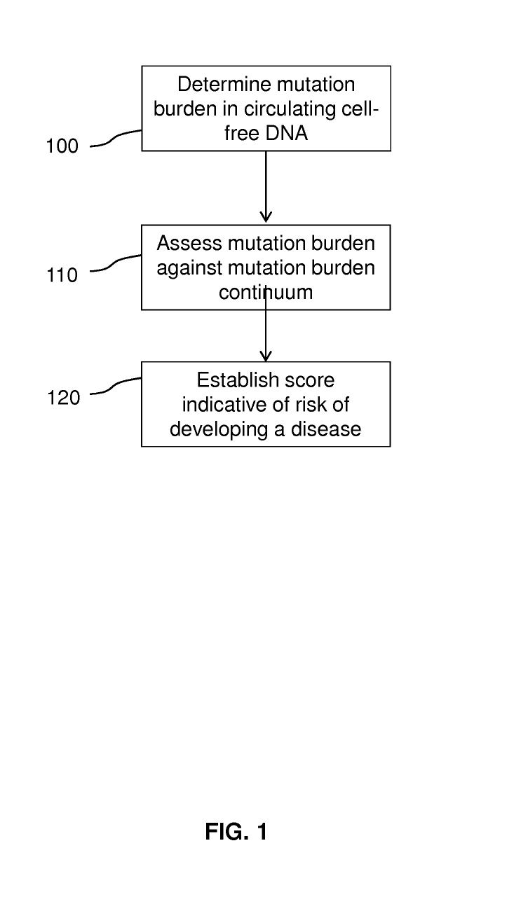

The invention generally relates to methods of assessing an individual's risk of developing a disease by determining mutation burden in circulating cell-free nucleic acid. In one embodiment, the invention relates to establishing a score indicative of the individual's risk by assessing the individual's mutation burden against a mutation burden continuum based on data from individuals with known clinical outcomes and mutation burdens. The value of the score in predicting disease risk may be assessed in multivariate models incorporating known clinical, genetic, behavioral, and environmental determinants of risk. A mutation burden continuum may contain various threshold scores that may have utility in guiding specific interventions (e.g., screening by conventional methods), which may also be specified based on the modeling. The reference sequence and the mutation burden continuum may be determined from a variety of sources. In certain aspects, methods of the invention relate to compilation of a database of mutations for individuals along with characteristics for each individual, which can include clinical and pathologic features (e.g., age, gender, comorbid diseases, family history), as well as behavioral and environmental exposures (e.g., diet, exercise, or smoking). FIG. 1 illustrates an exemplary embodiment of certain methods according to the invention, including the steps of determining a mutation burden in an individual's circulating cell-free DNA 100; assessing that mutation burden against a mutation burden continuum 110; and establishing a score indicative of the individual's risk of developing a disease 120.

### Nucleic Acid Isolation

In certain embodiments, circulating cell-free nucleic acid is obtained from an individual. Circulating cell-free nucleic acid may be any fragments of DNA or ribonucleic acid (RNA) that are present in the blood of an individual. Cell-free nucleic acid may be from sub-cellular sources such as mitochondria or other organelles or cell fragments from any cell type in the human body. In a preferred embodiment, the circulating cell-free nucleic acid is one or more fragments of DNA obtained from the plasma or serum of the individual.

The circulating cell-free nucleic acid may be isolated according to techniques known in the art and include, for example, the QIAmp system from Qiagen (Venlo, Netherlands), the Triton/Heat/Phenol protocol (THP) (Xue, et al., Optimizing the Yield and Utility of Circulating Cell-Free DNA from Plasma and Serum”, Clin. Chim. Acta., 2009; 404(2): 100-104), blunt-end ligation-mediated whole genome amplification (BL-WGA) (Li, et al., “Whole Genome Amplification of Plasma-Circulating DNA Enables Expanded Screening for Allelic Imbalance in Plasma”, J. Mol Diagn. 2006 February; 8(1): 22-30), or the NucleoSpin system from Macherey-Nagel, GmbH & Co. KG (Duren, Germany). In an exemplary embodiment, a blood sample is obtained from the individual and the plasma is isolated by centrifugation. The circulating cell-free nucleic acid may then be isolated by any of the techniques above.

According to certain embodiments of the invention, nucleic acid for a reference sequence determination may be extracted from somatic cells obtained from the individual by, for example, buccal swab. In certain aspects, white blood cells of the individual may be used as somatic cells according to the invention. The nucleic acids may be extracted through cell lysis. Lysing of the cells can be performed by methods known in the art. After cells have been obtained from the individual, it is preferable to lyse cells in order to isolate nucleic acids. Lysing methods may include sonication, freezing, boiling, exposure to detergents, or exposure to alkali or acidic conditions. The concentration of the detergent can be up to an amount where the detergent remains soluble in the solution. The detergent, particularly one that is mild and non-denaturing, can act to solubilize the sample. Detergents may be ionic or nonionic. Examples of nonionic detergents include triton, such as the Triton® X series (Triton® X-100 t-Oct-C6H4-(OCH2-CH2)xOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl) dimethyl-ammonio]-1-propanesulfonate. It is contemplated also that urea may be added with or without another detergent or surfactant.

Lysis or homogenization solutions may further contain other agents, such as reducing agents. Examples of such reducing agents include dithiothretol (DTT), f3-mercaptoethanol, DTE, GSH, cysteine, cystemine, tricarboxyethyl phosphine (TCEP), or salts of sulfurous acid.

Extracted nucleic acids may be further separated by e.g., differential precipitation, column chromatography, electrophoresis, or extraction with organic solvents. Extracts may then be further treated, for example, by filtration and/or centrifugation and/or with chaotropic salts such as guanidinium isothiocyanate or urea or with organic solvents such as phenol and/or HCCl₃ to denature any contaminating and potentially interfering proteins. The nucleic acid can also be resuspended in a hydrating solution, such as an aqueous buffer. The nucleic acid can be suspended in, for example, water, Tris buffers, or other buffers. In certain embodiments the nucleic acid can be re-suspended in Qiagen DNA hydration solution, or other Tris-based buffer of a pH of around 7.5.

Depending on the type of method used for extraction, the nucleic acid obtained can vary in size. The integrity and size of nucleic acid can be determined by pulse-field gel electrophoresis (PFGE) using an agarose gel.

### Nucleic Acid Amplification

Certain aspects of the invention utilize amplification of the cell-free circulating or somatic cell extracted nucleic acid in order to increase the copies of genetic material available for sequencing analysis. Amplification methods include, for example, amplification of a single target nucleic acid and multiplex amplification (amplification of multiple target nucleic acids in parallel). Amplification refers to production of additional copies of a nucleic acid sequence and is generally conducted using polymerase chain reaction (PCR) or other technologies well-known in the art (e.g., Dieffenbach and Dveksler, PCR Primer, a Laboratory Manual, 1995, Cold Spring Harbor Press, Plainview, N.Y.). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany, F. Genome research, 1:5-16 (1991); Barany, F., PNAS, 88:189-193 (1991); U.S. Pat. No. 5,869,252; and U.S. Pat. No. 6,100,099), strand displacement amplification and restriction fragment length polymorphism, transcription based amplification system, rolling circle amplification, and hyper-branched rolling circle amplification. Further examples of amplification techniques that can be used include, without limitation, quantitative PCR, quantitative fluorescent PCR (QF-PCR), multiplex fluorescent PCR (MF-PCR), real time PCR (RTPCR), single cell PCR, restriction fragment length polymorphism (PCR-RFLP), RT-PCR-RFLP, hot start PCR, in situ polonony PCR, in situ rolling circle amplification (RCA), bridge PCR, picotiter PCR, and emulsion PCR. Other suitable amplification methods include transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In certain embodiments, the amplification reaction is the polymerase chain reaction. Polymerase chain reaction refers to methods by K. B. Mullis (U.S. Pat. Nos. 4,683,195 and 4,683,202, hereby incorporated by reference) for increasing concentration of a segment of a target sequence in a mixture of nucleic acid without cloning or purification.

Multiplex polymerase chain reaction (Multiplex PCR) is another modification of polymerase chain reaction and is used in order to rapidly detect multiple gene sequences in a single PCR reaction. Multiplex PCR is typically accomplished using multiple primer sequences, each with a unique fluorophore for detection and quantification. This process amplifies DNA samples using the primers along with temperature-mediated DNA polymerases in a thermal cycler. Multiplex-PCR consists of multiple primer sets within a single PCR mixture to produce amplicons that are specific to different DNA sequences. In certain aspects, whole genome amplification may be accomplished through the random-primer initiated multiple displacement amplification

Typically, as much as 5-plex real-time qPCR is achievable in a PCR mixture by using fluorescently labeled probes, each one corresponding to a unique DNA sequence, which when amplified by a DNA polymerase, emit a fluorescence signal at its specified spectral wavelength. The spectral frequency discrimination between different fluorophores, or reporters, attached to each probe sequence enables detection of up to five different amplicon sequences, one for each fluorescent color that can be identified. Multiplexing beyond 5-plex is difficult due to insufficient spectral wavelengths that can be optically distinguished using current state of the art fluorescence excitation and emission filter sets.

Multiplex amplification strategies may be used analytically, as in detection methodologies, or preparatively, often for next-generation sequencing or other sequencing techniques. In the preparative setting, the output of an amplification reaction is generally the input to a shotgun library protocol, which then becomes the input to the sequencing platform. The shotgun library is necessary in part because next-generation sequencing yields reads significantly shorter than amplicons such as exons.

Amplification or sequencing adapters or barcodes, or a combination thereof, may be attached to a fragmented nucleic acid molecule. Such molecules may be commercially obtained, such as from Integrated DNA Technologies (Coralville, Iowa). In certain embodiments, such sequences are attached to the template nucleic acid molecule with an enzyme such as a polymerase or ligase. Suitable ligases include T4 DNA ligase and T4 RNA ligase, available commercially from New England Biolabs (Ipswich, Mass.). The ligation may be blunt ended or via use of complementary overhanging ends. In certain embodiments, following fragmentation, the ends of the fragments may be repaired, trimmed (e.g., using an exonuclease), or filled (e.g., using a polymerase and dNTPs) to form blunt ends. In some embodiments, end repair is performed to generate blunt end 5′ phosphorylated nucleic acid ends using commercial kits, such as those available from Epicentre Biotechnologies (Madison, Wis.). Upon generating blunt ends, the ends may be treated with a polymerase and dATP to form a template independent addition to the 3′-end and the 5′-end of the fragments, thus producing a single A overhanging. This single A can guide ligation of fragments with a single T overhanging from the 5′-end in a method referred to as T-A cloning. Alternatively, because the possible combination of overhangs left by the restriction enzymes are known after a restriction digestion, the ends may be left as-is, i.e., ragged ends. In certain embodiments double stranded oligonucleotides with complementary overhanging ends are used.

In certain applications, one or more barcode is attached to each, any, or all of the fragments. A barcode sequence generally includes certain features that make the sequence useful in sequencing reactions. The barcode sequences are designed such that each sequence is correlated to a particular portion of nucleic acid, allowing sequence reads to be correlated back to the portion from which they came. Methods of designing sets of barcode sequences are shown for example in U.S. Pat. No. 6,235,475, the content of which is incorporated by reference herein in its entirety. In certain embodiments, the barcode sequences range from about 5 nucleotides to about 15 nucleotides. In a particular embodiment, the barcode sequences range from about 4 nucleotides to about 7 nucleotides. In certain embodiments, the barcode sequences are attached to the template nucleic acid molecule, e.g., with an enzyme. The enzyme may be a ligase or a polymerase, as discussed above. Attaching barcode sequences to nucleic acid templates is shown in U.S. Pub. 2008/0081330 and U.S. Pub. 2011/0301042, the content of each of which is incorporated by reference herein in its entirety. Methods for designing sets of barcode sequences and other methods for attaching barcode sequences are shown in U.S. Pat. Nos. 6,138,077; 6,352,828; 5,636,400; 6,172,214; 6,235,475; 7,393,665; 7,544,473; 5,846,719; 5,695,934; 5,604,097; 6,150,516; RE39,793; 7,537,897; 6172,218; and 5,863,722, the content of each of which is incorporated by reference herein in its entirety. After any processing steps (e.g., obtaining, isolating, fragmenting, amplification, or barcoding), nucleic acid can be sequenced.

### Nucleic Acid Sequencing

In various aspects, methods of the invention relate to sequencing of nucleic acid samples isolated from somatic cells of the individual or the sequencing of circulating cell-free nucleic acid. Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, translocation through a nanopore or nanochannel, digestion or polymerization of DNA combined with detection of nucleotides in a nanopore or nanochannel, optical detection of nucleotides in strands localized with a nanopore or nanochannel, and SOLiD sequencing. Separated molecules may be sequenced by sequential or single extension reactions using polymerases or ligases as well as by single or sequential differential hybridizations with libraries of probes.

In some embodiments, a sequencing technique (e.g., a next-generation sequencing technique) is used to sequence part of one or more captured targets (e.g., or amplicons thereof) and the sequences are used to count the number of different barcodes that are present. Accordingly, in some embodiments, aspects of the invention relate to a highly-multiplexed qPCR reaction.

A sequencing technique that can be used includes, for example, Illumina sequencing. Illumina sequencing is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pat. No. 7,960,120; U.S. Pat. No. 7,835,871; U.S. Pat. No. 7,232,656; U.S. Pat. No. 7,598,035; U.S. Pat. No. 6,911,345; U.S. Pat. No. 6,833,246; U.S. Pat. No. 6,828,100; U.S. Pat. No. 6,306,597; U.S. Pat. No. 6,210,891; U.S. Pub. 2011/0009278; U.S. Pub. 2007/0114362; U.S. Pub. 2006/0292611; and U.S. Pub. 2006/0024681, each of which is incorporated by reference in their entirety.

Sequencing generates a plurality of reads. Reads generally include sequences of nucleotide data less than about 150 bases in length, or less than about 90 bases in length. In certain embodiments, reads are between about 80 and about 90 bases, e.g., about 85 bases in length. In some embodiments, these are very short reads, i.e., less than about 50 or about 30 bases in length.

A sequencing technique that can be used in the methods of the provided invention includes, for example, 454 sequencing (454 Life Sciences, a Roche company, Branford, Conn.) (Margulies, M et al., Nature, 437:376-380 (2005); U.S. Pat. No. 5,583,024; U.S. Pat. No. 5,674,713; and U.S. Pat. No. 5,700,673). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology by Applied Biosystems from Life Technologies Corporation (Carlsbad, Calif.). In SOLiD sequencing, DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that can be used in the methods of the provided invention is Ion Torrent sequencing, described, for example, in U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the content of each of which is incorporated by reference herein in its entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (H.sup.+), which signal is detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in U.S. Pub. 2011/0009278, U.S. Pub. 2007/0114362, U.S. Pub. 2006/0024681, U.S. Pub. 2006/0292611, U.S. Pat. No. 7,960,120, U.S. Pat. No. 7,835,871, U.S. Pat. No. 7,232,656, U.S. Pat. No. 7,598,035, U.S. Pat. No. 6,306,597, U.S. Pat. No. 6,210,891, U.S. Pat. No. 6,828,100, U.S. Pat. No. 6,833,246, and U.S. Pat. No. 6,911,345, each of which are herein incorporated by reference in their entirety.

Another example of a sequencing technology that can be used in the methods of the provided invention includes the single molecule, real-time (SMRT) technology of Pacific Biosciences (Menlo Park, Calif.). In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni, G. V., and Meller, A., Clin Chem 53: 1996-2001 (2007)). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in U.S. Pub. 2009/0026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M., PNAS, 53:564-71(1965)). In one example of the technique, individual DNA molecules are labeled using metallic labels that are distinguishable using an electron microscope. These molecules are then stretched on a flat surface and imaged using an electron microscope to measure sequences.

Another example of a sequencing technique that can be used in the methods of the provided invention involves Fast Aneuploidy Screening Test-Sequencing System (FAST-SeqS), as described in PCT application PCT/US2013/033451, which is incorporated by reference. See also Kinde et al., “FAST-SeqS: A Simple and Efficient Method for the Detection of Aneuploidy by Massively Parallel Sequencing,” DOI: 10.1371/journal.pone.0041162, which is incorporated by reference. FAST-SeqS uses specific primers, specifically, a single pair of primers that anneal to a subset of sequences dispersed throughout the genome. The regions are selected due to similarity so that they could be amplified with a single pair of primers, but sufficiently unique to allow most of the amplified loci to be distinguished. FAST-SeqS yielded sequences align to a smaller number of positions, as opposed to traditional whole genome amplification libraries in which each tag must be independently aligned.

Sequence assembly can be accomplished by methods known in the art including reference-based assemblies, de novo assemblies, assembly by alignment, or combination methods. In some embodiments, sequence assembly uses the low coverage sequence assembly software (LOCAS) tool described by Klein, et al., in LOCAS-A low coverage sequence assembly tool for re-sequencing projects, PLoS One 6(8) article 23455 (2011), the contents of which are hereby incorporated by reference in their entirety. Sequence assembly is described in U.S. Pat. No. 8,165,821; U.S. Pat. No. 7,809,509; U.S. Pat. No. 6,223,128; U.S. Pub. 2011/0257889; and U.S. Pub. 2009/0318310, the contents of each of which are hereby incorporated by reference in their entirety.

### Determining Mutation Burden

Methods of the invention include determining mutation burden in circulating cell-free nucleic acid in order to predict the likelihood of disease or disease recurrence. In certain embodiments, mutation burden is determined by identifying the number of mutations present in the individual's circulating cell-free nucleic acid sequence compared to a reference nucleic acid sequence. In some aspects, the determination of mutation burden may reflect the amount or level of each mutation identified in the individual's cfDNA (i.e. the allelic fraction in cfDNA). Sequence data for the individual's circulating cell-free nucleic acid sample may be determined using the techniques described above. In certain aspects, a reference nucleic acid sequence may be determined through isolating and sequencing a nucleic acid sample from any somatic cell of the individual. In a preferred embodiment, a reference nucleic acid sequence is obtained by isolating, amplifying, and sequencing nucleic acid obtained from a somatic cell source, such as a white blood cell, or buccal swab of the individual at the same time as the blood or urine sample containing circulating cell-free nucleic acid is obtained. In certain aspects, a plurality of cell types from a plurality of locations in the individual's body may be used as sources of somatic cell nucleic acids. In certain embodiments, sequence information from these various nucleic acids along with the circulating cell-free nucleic acid may be compared in order to determine a nucleotide base variance across the multiple nucleic acid samples. In these embodiments, the mutation burden for the individual may be calculated from this variance.

In certain aspects, the reference nucleic acid sequence is a nucleic acid sequence from any cell or circulating cell-free nucleic acid that has been obtained from the individual, isolated, amplified, and/or sequenced at an earlier time. For example, a sequence of DNA obtained from a buccal swab or white blood cell of the individual during adolescence may then be used throughout the individual's life as a non-mutated reference from which to determine present day mutation burden. In certain aspects, the reference sequence may be determined from the individual's cell-free nucleic acid. For example, where there is variability among multiple sequencing reads of the same section of an individual's cell-free nucleic acid sample, a reference sequence may be determined from the most prevalently represented sequence variation at any given locus of the individual's cell-free nucleic acid sequence. In certain aspects, the frequency of occurrence of a particular mutation among multiple sequencing reads of the same region of an individual's cell-free nucleic acid may be used to determine the allelic fraction or level of a mutation. A higher frequency of a mutation may indicate somatic mosaicism and may be assigned a higher mutation burden. In certain embodiments, an individual mutation may be weighted in determining mutation burden according to the frequency of occurrence of the mutation.

In other embodiments, the reference may simply be the known human genome sequence (non-mutated) with a mutation frequency estimated by the average frequency in a large population of unaffected individuals. In such embodiments, mutations present at or above a threshold rate in the sample population may be considered germline variability, as opposed to somatic mutations. In various embodiments, this threshold rate of mutation occurrence may be determined based on the size of the sample population and may be, for example, 10%, 20%, 30%, 40%, or 50%.

Determining mutation burden of circulating cell-free nucleic acid sequence is accomplished by comparing it to the reference sequence and may include alignment of two or more sequences using, for example, a matching algorithm.

Determination of mutation burden may include, for example, identification of the type of mutation, the location in the genome of the mutation, and/or the frequency or level of the mutation within the individual's cells. This information may be used to weight various mutations in accordance with their impact or potential impact on disease. For example, known oncogenic mutations such as EGFR L858R or BRAF V600E may be weighted higher than: mutations in genes known to be related to cancer but with less frequent involvement in cancer; mutations in genes without known links to disease; or mutations in tumor suppressor genes which may be mutated in healthy cells, since a single working copy of the gene can allow the cell to function properly.

### Establishing a Risk Score

A risk score indicative of risk of developing a disease or of disease recurrence may be established for an individual by assessing the individual's mutation burden on a mutation burden continuum which may contain thresholds known or empirically determined to be associated with the likelihood of disease. Alternatively, a risk score may be used longitudinally in order to assess the rate of change in mutation burden in an individual over time. The risk score may be used to advise the individual on whether or not to seek additional testing, to participate in a screening regimen for a particular disease, or to contemplate lifestyle changes to reduce the rate of mutation burden accumulation. In certain embodiments, the risk score may be used to avoid unnecessary monitoring, thereby saving money and risk from unnecessary procedures. An exemplary algorithm for determining the risk score may include, for example, the following:

RF=4000*(# activating oncogene mutations)+500*(#loss of function tumor suppressor gene mutations)+50*(#total mutations identified across all loci)

For reporting of risk for an individual, output from a multivariate model incorporating the risk score as well as other contributory factors (e.g. age, family history, smoking history) may be used. The risk factor algorithm can be calculated alongside a cost function to allow risk determination at the lowest possible assay cost. The algorithm may include non-linear terms, terms specific to individual mutations, exponential or logarithmic terms, and the like. For any specific disease, the algorithm may be derived through development and validation of a model constructed on a training set of clinical data followed by validation on an independent data set, an example of which could consist of a non-negative least squares fit of a function where the multipliers and exponents of each risk variables are free parameters, and the optimization fit is conducted to achieve the highest correlation, in a training data set, between the risk factor metric as calculated by the evolving algorithm, and the disease state of the training set samples, while minimizing a function describing the real cost of testing each mutation or variable in an individual. In another embodiment, the algorithm may take the form of a Hidden Markov Model or neural network trained on a set of training data composed of a range of normal and disease samples. In another embodiment, Bayesian methods may be used to train an algorithm on a training data set.

The mutation burden continuum used in establishing a risk score may be developed from a variety of sources. The continuum may contain average mutation burdens for various sample populations or for an individual at multiple time points. A sample population may be defined by one or more characteristics including, age, sex, race, geographic location, disease state, weight, height, or other body measurements or health indicators.

In certain aspects, methods of the invention relate to creating a database of patient information including mutation burden and characteristics as described above. Such a database may be used to develop a mutation burden continuum in certain embodiments of the invention. Additionally the database may be used to identify and/or track concentrations of high mutation burden based on individual characteristics. The database may be created using a computing system as described below. Records of mutation burden and disease state for an individual over time can be used according to methods of the invention to calculate risk scores for individuals with similar mutation burdens of developing similar diseases.

According to methods of the invention, the mutation burden for an individual may be recorded over numerous time points. Thus, a record of mutation burden may be used to track the accumulation of mutations over time and/or the change in rate of accumulation. The rate of change may itself be a risk score for disease or disease recurrence. In certain aspects, one or more of the individual's prior mutation burdens may be part of the mutation burden continuum wherein the individual's risk score is determined by comparing the present mutation burden to the individual's prior mutation burden. A chronological record of the individual's mutation burden may be used to identify sources of exposure to mutagenic stress or to identify deterioration in the nucleic acid repair mechanisms in the individual's cells.

As one skilled in the art recognizes as necessary or best-suited for performance of the methods of the invention, including comparison of the cell-free nucleic acid sequence and the reference nucleic acid sequence as well as assignment of severity factors and calculation or weighting of mutation burden may include one or more computing systems that include one or more of a processor (e.g., a central processing unit (CPU), a graphics processing unit (GPU), etc.), a computer-readable storage device (e.g., main memory, static memory, etc.), or combinations thereof which communicate with each other via a bus.

A processor may include any suitable processor known in the art, such as the processor sold under the trademark XEON E7 by Intel (Santa Clara, Calif.) or the processor sold under the trademark OPTERON 6200 by AMD (Sunnyvale, Calif.).

Memory preferably includes at least one tangible, non-transitory medium capable of storing: one or more sets of instructions executable to cause the system to perform functions described herein (e.g., software embodying any methodology or function found herein or computer programs referred to above); data (e.g., images of sources of medication data, personal data, or a database of medications); or both. While the computer-readable storage device can, in an exemplary embodiment, be a single medium, the term “computer-readable storage device” should be taken to include a single medium or multiple media (e.g., a centralized or distributed database, and/or associated caches and servers) that store the instructions or data. The term “computer-readable storage device” shall accordingly be taken to include, without limit, solid-state memories (e.g., subscriber identity module (SIM) card, secure digital card (SD card), micro SD card, or solid-state drive (SSD)), optical and magnetic media, and any other tangible storage media.

Any suitable services can be used for storage such as, for example, Amazon Web Services, memory of the computing system, cloud storage, a server, or other computer-readable storage.

Input/output devices according to the invention may include one or more of a display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, a button, an accelerometer, a microphone, a cellular radio frequency antenna, a network interface device, which can be, for example, a network interface card (NIC), Wi-Fi card, or cellular modem, or any combination thereof.

One of skill in the art will recognize that any suitable development environment or programming language may be employed to implement the methods described herein. For example, methods herein can be implemented using Perl, Python, C++, C#, Java, JavaScript, Visual Basic, Ruby on Rails, Groovy and Grails, or any other suitable tool. For a mobile device, it may be preferred to use native xCode or Android Java.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of assessing a risk of an individual developing a disease, comprising:
amplifying circulating cell-free nucleic acid obtained from the individual through whole genome amplification to obtain a first amplicon;
sequencing the first amplicon to obtain a cell-free sequence;
amplifying a cellular nucleic acid extracted from one or more somatic cells obtained from the individual to obtain a second amplicon;
sequencing the second amplicon to obtain a cellular sequence;
determining a number of mutations in the cell free sequence relative to the cellular sequence by comparing the cell-free sequence to the cellular sequence; and
applying a multivariate model to the number of mutations to produce an RF score, wherein the multivariate model comprises a weighted term for disease-associated mutations and a weighted term for total mutations with different weight values for each weighted term, wherein the RF
score is indicative of risk of the individual developing a disease.

2. The method of claim 1 wherein the circulating cell-free nucleic acid is isolated from urine, plasma, or serum of the individual.

3. (canceled)

4. The method of claim 1 wherein the reference sequence is a consensus sequence from an unaffected sample population.

5. The method of claim 4 wherein the determining step further comprises not counting a mutation present at an abundance of 50% or higher in the unaffected sample population.

6. (canceled)

7. The method of claim 1 wherein the somatic cell of the individual is obtained by a buccal swab of the individual.

8. The method of claim 6 wherein the somatic cells of the individual are the individual's white blood cells.

9. The method of claim 1 wherein the reference nucleic acid sequence is from a previously-obtained cell-free nucleic acid sample from the individual.

10. The method of claim 1 wherein the sequencing step comprises next generation sequencing (NGS).

11. The method of claim 1 wherein the mutations are selected from the group consisting of a loss of heterozygosity, a single nucleotide variant, a deletion, an insertion, a rearrangement, copy number change, and a translocation.

12. The method of claim 1 wherein establishing the risk score further comprises assessing the individual's number of mutations against a mutation burden continuum comprising one or more average numbers of mutations for one or more sample populations.

13. The method of claim 12 wherein the sample population is defined by one or more characteristics in common.

14. The method of claim 13 wherein the one or more characteristics are selected from the group consisting of age, sex, race, a geographic location, a disease state, weight, and height.

15. The method of claim 12 wherein the mutation burden continuum further comprises one or more threshold numbers of mutations associated with one or more risk levels of developing the disease.

16. The method of claim 1 further comprising:
creating a chronological record of the number of mutations of the individual, said chronological record comprising a plurality of prior numbers of mutations of the individual.

17. The method of claim 16 wherein establishing the risk score further comprises comparing a current number of mutations of the individual to one or more prior numbers of mutations of the individual.

18. The method of claim 16 further comprising calculating a rate of change in the number of mutations of the individual over time wherein an increase in the rate of change is indicative of a higher risk of the individual developing a disease.

19. The method of claim 1 wherein the disease is selected from the group consisting of cancer, a neurological disease, a cardiovascular disease, an autoimmune disorder, or a metabolic disease.

20. The method of claim 1 further comprising compiling a mutation burden database, said database comprising the number of mutations for a plurality of individuals and one or more characteristics for the plurality of individuals.

21. The method of claim 20 wherein the one or more characteristics are selected from the group consisting of age, sex, race, a geographic location, a disease state, weight, and height.

22. The method of claim 1 wherein the individual's number of mutations is weighted according to a severity factor assigned to one or more mutations in the individual's circulating cell-free nucleic acid.

23. The method of claim 1 wherein the determining step further comprises comparing the circulating cell-free nucleic acid sample sequence to the reference nucleic acid sequence to determine a frequency of occurrence for the somatic mutations in the individual's sample.

24. The method of claim 1 wherein the multivariate model comprises RF=400*(a number of activating oncogene mutations)+500*(a number of loss of function tumor suppressor gene mutations)+50*(a total number of mutations identified across all sequenced loci).

25. The method of claim 1 wherein the mutations are single nucleotide variants in the cell-free sequence.
